(19) United States
(12) Patent Application Publication
(10) Pub. No.: US 2022/0072287 A1
(43) Pub. Date: Mar. 10, 2022

(54) Butt Crack Swab

(71) Applicant: Isaac Mulaw, Oregon City, OR (US)

(72) Inventor: Isaac Mulaw, Oregon City, OR (US)

(21) Appl. No.: 17/013,853

(22) Filed: Sep. 7, 2020

(51) Int. Cl. A61F 13/40; A61F 13/15; A61F 13/45; A61F 13/53

(52) U.S. Cl. CPC A61M 35/006; A61F 13/15707; A61F 13/15617; A61F 13/45; A61F 13/53; A61M 2210/10; A61F 2013/530313; A61F 2013/15008; A61F 2013/15463; A61F 2013/530116; A61F 2013/530145; A61F 2013/530197; A61M 2205/02

(57) ABSTRACT

A butt crack swab with a pressed molded fiber substrate, a conformal flushable wipe and a binder material. The pressed molded fiber substrate is shaped to fit within the intergluteal cleft of a person and in close proximity to the cluneal cleft. The conformal flushable wipe is fused on top of the molded fiber substrate via the binder material. A preferred embodiment includes the pressed molded fiber substrate made of recyclable material.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of medicated, and absorbent swabs and more specifically to a butt crack swab and to an armpit swab.

In certain cases it is necessary for a person or a medical assistant to apply topical medications on the area medically known as the intergluteal cleft, or commonly known as the butt crack and specifically in the area close to the anus known as the cluneal cleft which is the groove between the buttocks that runs from just below the sacrum to the perineum, so named because is forms the visible border between the external rounded protrusions of the gluteus maximus muscles. Medical wipes, such as “NatureSoft Flushable Cleansing Dry Wipes” made by Medline Corporation, have been used for applying ointments, creams or gels.

However, it is difficult to put a cream or ointment onto a swab in a consistent and controlled manner. Additionally, if the swab is meant to be held in place for a period of time, the flat shape of a typical swab or wipe is not ideal to accommodate the compound curved nature of the application area.

### BRIEF SUMMARY OF THE INVENTION

The primary object of the invention is to provide a biodegradable flushable swab, infused with medication, or simply acting as a moisture absorber, that can fit comfortably for a period of time in the intergluteal cleft and in close contact with the cluneal cleft.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed a butt crack swab comprising: a pressed molded fiber substrate, a conformal flushable wipe or swab sheet, a binder material, said pressed molded fiber substrate shaped to fit within the intergluteal cleft of a person and in close proximity to the cluneal cleft, and said conformal flushable wipe sheet fused onto said molded fiber substrate via said binder material.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a perspective view of the embodiment.

FIG. 2 is side view of the embodiment.

FIG. 3 is a perspective view of the embodiment in the curved position.

FIG. 4 is a section view of a person wearing the embodiment.

FIG. 5 is an exploded view of the embodiment.

FIG. 6 is a side section view of the embodiment.

FIG. 7 is a perspective view of the lower layer in the curved position.

FIG. 8 is a front view of a person wearing an armpit swab embodiment of the invention.

FIG. 9 is a perspective view of the armpit absorber in the folded position.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

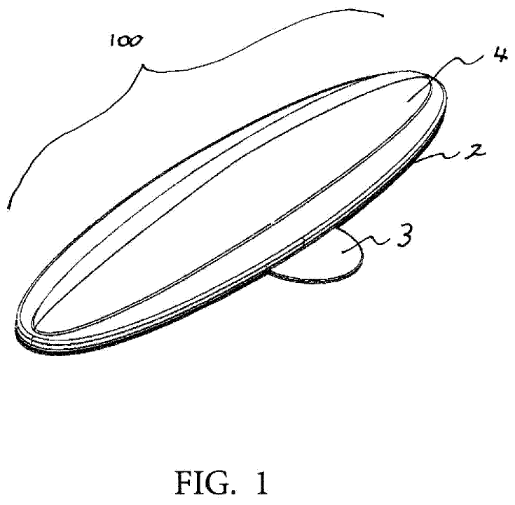

Referring now to FIG. 1 we see a perspective view of the butt swab 100 of the present embodiment. The swab 100 has a perimeter that is elliptical in shape. An under layer 2 is a substrate made of flushable, biodegradable pressed paper fiber material. The upper layer 4 conforms to the lower layer 2 and is made of flushable, biodegradable super absorbent polymer, otherwise known as SAP, such as fibrous polypropylene. Another material that has recently been tested by the Indian Institute of Technology is a new SAP material made of electrospun cellulose acetate nanofibers which is claimed to be even more flushable and biodegradable than current SAPs. A standard binder 12, usually made from vegetable starch, joins the two layers 2,4 together. The entire assembly 100 has a longitudinally oriented raised portion whose shape is designed to comfortably fit between the users intergluteal cleft. The upper layer 4 is thick enough to allow for the introduction and retention of various creams, gels or ointments that may come from medical manufacturers in prepackaged in airtight pouches. Alternately, the swab can be sold without additives and the end user can apply medication to the swab 100 and use the swab 100 to either wipe the area in proximity to the cluneal cleft or have the swab 100 remain in place for an extended period of time to let a medication have an on-going effect. Another option is to apply the swab 100 in a dry form and leave it there for an extended period to allow it to absorb excess moisture that may accumulate in the intergluteal cleft area. The under layer 2 has enough dimensional stability to allow the longitudinal raised portion to retain its shape, but to be flexible enough for the entire invention 100 to be curved to fit the contour of the cluneal cleft. An optional tab 3 can be used to help pull the butt swab 100 from the user's intergluteal cleft. The tab 3 is shown in FIG. 1 as an option, however the remaining Figures do not show it.

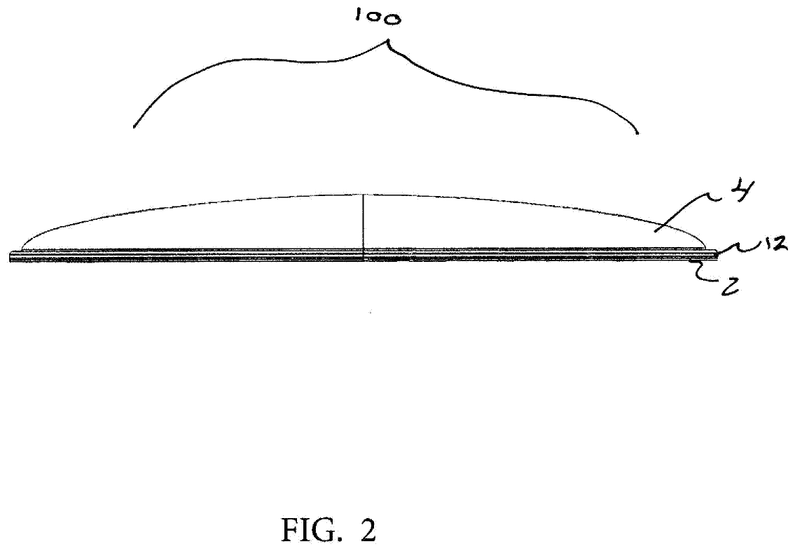

FIG. 2 is a side view of the invention 100 clearly showing how the longitudinal raised portion of the upper layer 4 starts from a low point at either end, and forms a high point at the center.

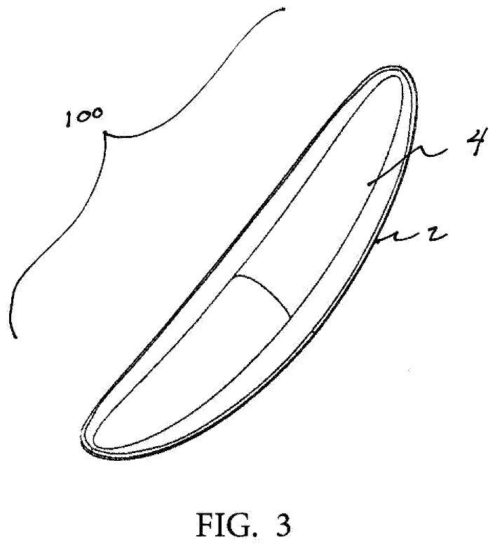

FIG. 3 is a perspective view of the invention 100 bent into a curved format that is ready to accommodate the curve of the cluneal cleft.

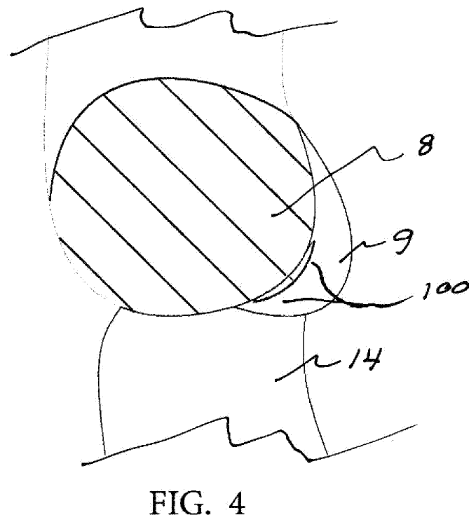

FIG. 4 is a partial perspective view of a person with one leg removed 8 to show the location of the butt swab 100 in the cluneal cleft with right butt cheek 9 visible.

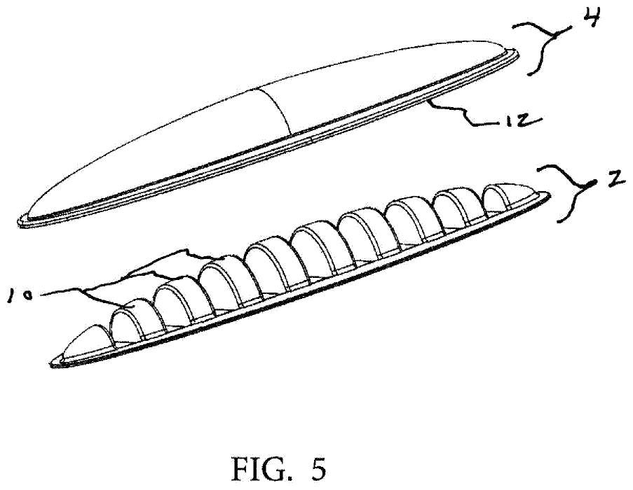

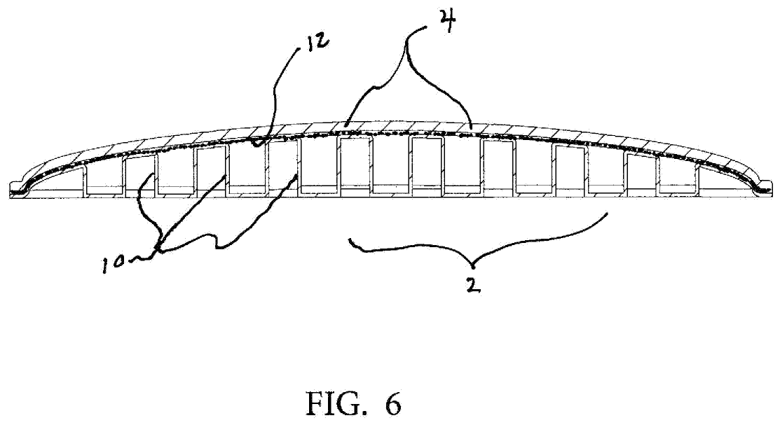

FIG. 5 is an exploded view showing the bottom layer 2 and the top layer 4. The bottom layer 2 is made of recycled paper fiber that can hold its shape, but is flexible enough to be conformable to the cluneal cleft and can also quickly melt and disintegrate when fully submerged in water, as in a standard toilet environment. The ribs 10 allow the layer 4 to bend but to still maintain the raised longitudinal configuration. The upper layer 4 is made of very flexible non-woven absorbent fibers as described above and is attached to the lower layer 2 by a standard binder 12 made from vegetable starch as shown in the side section view in FIG. 6.

FIG. 6 is a side section view of the invention 100. Layer 4 is glued to layer 2 via binder 12. Ribs 10 help maintain the raised longitudinal shape.

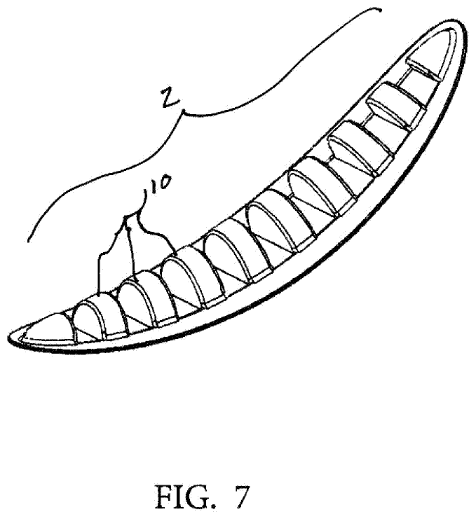

FIG. 7 is a perspective view of the lower layer 2 in its bent position showing the tops of ribs 10 being urged toward each other.

In the above described and illustrated way, the present butt swab of the present invention 100 can be used by individuals to apply medicinal ointments, creams or oils in a more controlled and effective way than with a standard flat absorbent pad. Additionally, the invention, because of its unique shape, can comfortably remain, if so desired, in the cluneal cleft area for extended periods of time either to administer medication over a prolonged period of time, or simply to absorb excess moisture that can accumulate in the cluneal cleft area in some individuals.

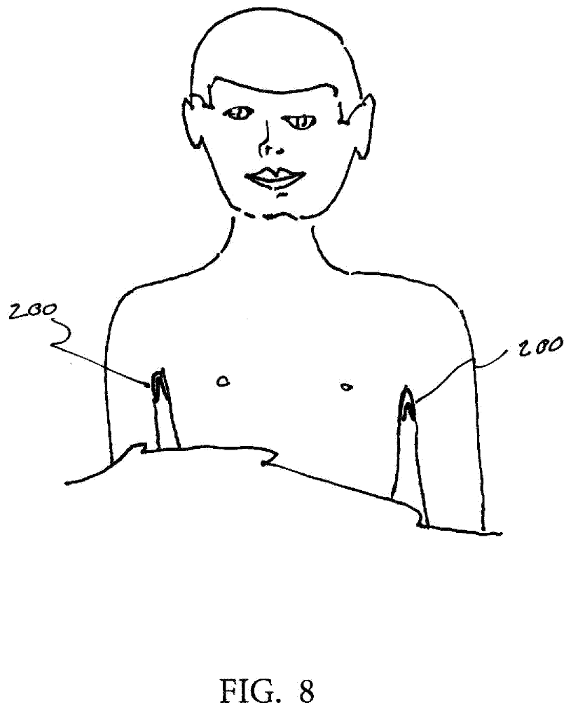

In an alternate embodiment 200 shown in FIG. 8, the pad 200 can be used in the armpit area of an individual to absorb sweat, to keep the area clean and dry, to prevent rashes, boils or itches, and to incorporate perspiration related odor controlling means, such as deodorants or creams, and medicated ointments.

The application of such alternate embodiment is very much appreciated by those who can't use current products due to physical limitations, such as the elderly or those with osteoarthritis or rheumatoid arthritis and the like; or those who prefer to use an alternative sweat control application means, especially while on the go, and those that have sensitive skin and need to avoid skin irritation from using current perspiration controlling means, such as deodorants.

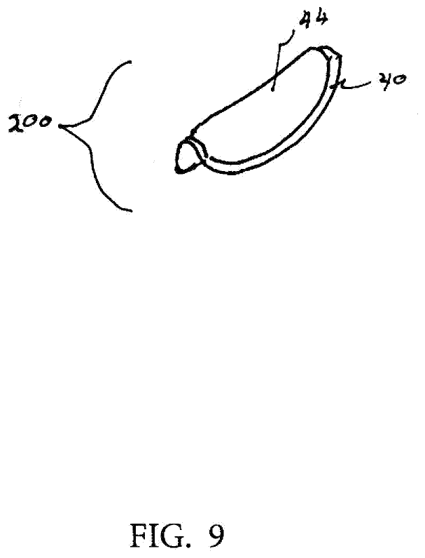

To hold the pad 200 in place, an adhesive perimeter surrounds the pad so that as the person raises his or her arms, the pad will remain in place. FIG. 8 is a front view of a person wearing the alternate embodiment 200. FIG. 9 is a perspective folded view of the alternate embodiment of the invention.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

We claim:

1. A biodegradable flushable device for delivery of medication to a person's cluneal cleft and for absorbing moisture from a person's cluneal cleft, wherein the device comprises:
a pressed molded fiber substrate comprising an elongated substantially elliptical shape, the elliptical shape comprising a major axis and a minor axis, the elliptical shape further comprising a flat outer perimeter and a gradually raised central portion peaking at the intersection of the major and of the minor axes;
a binder material; and
a conformal flushable wipe fused to the molded fiber substrate via said binder material.

2. A biodegradable flushable device of claim 1 wherein the pressed molded fiber substrate is made of recyclable paper material.

3. The biodegradable flushable device of claim 1 wherein the conformal flushable wipe is a sheet made of cellulose from wood pulp.

4. The biodegradable flushable device of claim 1 wherein conformal flushable wipe is made of electrospun cellulose acetate nanofibers.

5. The biodegradable flushable device of claim 1 wherein said binder material is dry roasted starch.

6. The biodegradable flushable device of claim 1 wherein the pressed molded elliptical shape is further shaped to fit within the intergluteal cleft of a person and in close proximity to the cluneal cleft.
